(19) United States
(12) Patent Application Publication
(10) Pub. No.: US 2003/0219730 A1
(43) Pub. Date: Nov. 27, 2003

(54) Base sequence characteristic of small round-structured

(75) Inventors: Kiyoshi Yasukawa, Kawasaki-shi (JP); Takahiko Ishiguro, Yokohama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: TOSOH CORPORATION, Shinnanyo-shi (JP)

(21) Appl. No.: 10/391,631

(22) Filed: Mar. 20, 2003

(30) Foreign Application Priority Data
Mar. 20, 2002 (JP) 2002-078971

(51) Int. Cl. C12Q 1/70; C07H 21/04

(52) U.S. Cl. 435/5; 536/23.72

(57) ABSTRACT

A cDNA sequence of any one of SEQ ID NOS:1 to 4 or a derivative thereof, which binds to a genogroup I small round-structured virus genome, or a fragment of a cDNA sequence of any one of SEQ ID NOS:1 to 4 or a derivative thereof, which is large enough to bind to a genogroup I small round-structured virus genome.

Small round structured viruses (SRSVs) are generally known as causative agents of viral food poisoning. The present invention relates to base sequences of SRSVs useful in clinical tests, hygienic tests, food tests and food poisoning tests.

Small round-structured viruses belong to the human Caliciviridae, which is divided into three genetic groups, genogroup I (GI), genogroup II (GII) and genogroup III (GIII). In general, GI and GII viruses are called SRSVs, while GIII viruses are called human caliciviruses (in a narrow sense).

Viruses are responsible for an estimated 20% of food poisoning cases reported in Japan. SRSVs are detected in over 80% of viral food poisoning cases. The leading infection source is food, and raw oysters are often problematic. Detection of SRSVs from infants and young children with (sporadic) acute gastroenteritis indicates transmissibility from human to human. Therefore, tests for SRSVs are an important issue in view of public hygiene and quality control of food.

To date, detection of SRSVs has been based on electron microscopy. However, this technique requires at least 10⁶ virus particles/ml to be present for successful detection and is limited to fecal specimens from patients. Besides, it is useful only for virus detection, but not for virus identification. The recent success in production of human calicivirus virus-like particles has triggered research for ELISA using them for detection of human calicivirus-specific antibodies in serum. However, it is no more sensitive than electron microscopy. Thus, conventional techniques are operationally complicated and time-consuming and can not achieve quick detection of traces of SRSVs in specimens.

Under the circumstances described above, the present inventors have proposed oligonucleotides that bind to intramolecularly free regions in the genomic RNAs of GI and GII SRSVs at relatively low and constant temperatures (35-50° C., preferably 41° C.) and oligonucleotide primers and oligonucleotide probes for amplification-based detection of SRSV RNA. Further, the present inventors have proposed a method of detecting GI and GII SRSVs using isothermal RNA amplification (Japanese Patent Application JP2000-182326, Japanese Patent Application JP2000-359482 and Japanese Patent Application JP2001-020231). However, GI and GII SRSVs are further divided into various subtypes. Furthermore, the variety of base sequences are rich between strains both for GI and GII SRSVs. Base sequences of many strains are needed to evaluate the above-mentioned detection techniques and develop new techniques for detecting GI or GII SRSV with high coverage, but base sequences that have been known to date is limited.

To solve this problem, the present inventors have identified the base sequences of regions containing the regions amplified for detection of four GI SRSVs and ten GII SRSVs by the above-mentioned method by trial and error PCR using oligonucleotides. Namely, the present invention provides base sequences characteristic of small round-structured viruses. In order to attain the above-mentioned object, the present invention defined in claim 1 of the present application provides a cDNA sequence of any one of SEQ ID NOS;1 to 4 or a derivative thereof, which binds to a GI SRSV genome, or a fragment of a cDNA sequence of any one of SEQ ID NOS:1 to 4 or a derivative thereof, which is large enough to bind to a genogroup, small round-structured virus genome. The present invention defined in claim 2 of the present application provides a cDNA sequence of any one of SEQ ID NOS:5 to 14 or a derivative thereof, which binds to a GII SRSV genome, or a fragment of a cDNA sequence of any one of SEQ ID NOS:5 to 14 or a derivative thereof, which is large enough to bind to a genogroup II small round-structured virus genome. Now, the present invention will be described in detail.

An oligonucleotide selected based on the base sequences of the present invention may be used in any gene amplification methods without any particular restrictions, including DNA amplification methods such as PCR and RNA amplification methods such as NASBA and 3SR.

The base sequence of the above-mentioned oligonucleotide is not particularly limited. Screening for such gene sequences is exemplified in Japanese Patent Application JP2000-182326, Japanese Patent Application JP2000-359482 and Japanese Patent Application JP2001-020231, which disclose screening for oligonucleotide sequences hybridizable specifically to intramolecularly free regions in a target RNA.

There is no particular restriction on how to use the oligonucleotide thus obtained for gene amplification. It may be used, for example, as a scissor probe for RNA cleavage, an oligonucleotide primer for nucleic acid amplification, or as a probe after partial modification or attachment of a detectable label. Its use for isothermal RNA amplification is described specifically in Japanese Patent Application JP2000-182326 and Japanese Patent Application JP2000-359482.

Now, the present invention will be described in further detail by referring to Examples. However, the present invention is by no means restricted to these specific Examples.

### EXAMPLE 1

### Base Sequencing

RNAs were extracted from four fecal specimens from four GI SRSV-infected patients and ten fecal specimens from ten GII SRSV-infected patients by an ordinary method. For the GI SRSVs, to select the base sequences of SEQ ID NOS:1 and 2, PCR amplification using an oligonucleotide of SEQ ID NO:15 and an oligonucleotide of SEQ ID NO:16 was followed by sequencing of the resulting DNAs. To select the base sequences of the base sequences of SEQ ID NOS:1 and 2, PCR amplification using an oligonucleotide of SEQ ID NO:17 and an oligonucleotide of SEQ ID NO:18 was followed by sequencing of the resulting DNAs.

For the GII SRSVs, to select the base sequences of SEQ ID NOS:5 to 10, PCR amplification using an oligonucleotide of SEQ ID NO:19 and an oligonucleotide of SEQ ID NO:20 was followed by sequencing of the resulting DNAs. To select the base sequences of SEQ ID NOS:11 to 14, PCR amplification using an oligonucleotide of SEO ID NO:21 and an oligonucleotide of SEQ ID NO:19 was followed by sequencing of the resulting DNAs.

As explained above, the base sequences of the present invention make it possible to develop oligonucleotides hybridizable to RNAs of genes of various GI SRSV strains and GII SRSV strains, which are useful for amplification and detection of SRSV genes.

The fragments of cDNA sequences of the present invention may be oligonucleotides consisting of at least 10 bases in the base sequences in the Sequence Listing. It is obvious from the fact base sequences of about 10 bases are sufficient to ensure for primers or probes the specificity for a target nucleic acid.

The entire disclosure of Japanese Patent Application No. 2002-78971 filed on Mar. 20, 2002 including specification, claims and summary is incorporated herein by reference in its entirety.

1. A CDNA sequence of any one of SEQ ID NOS:L to 4 or a derivative thereof, which binds to a genogroup I small round-structured virus genome, or a fragment of a CDNA sequence of any one of SEQ ID NOS:1 to 4 or a derivative thereof, which is large enough to bind to a genogroup I small round-structured virus genome.

2. A cDNA sequence of any one of SEQ ID NOS:5 to 14 or a derivative thereof, which binds to a genogroup II small round-structured virus genome, or a fragment of a cDNA sequence of any one of SEQ ID NOS:5 to 14 or a derivative thereof, which is large enough to bind to a genogroup II small round-structured virus genome.


SEQUENCE LISTING (20 sequences)

SEQ ID NO: 1 - DNA, 658 bp, Human calicivirus
ccaatgcaattgaacaggtcatggacacaccagtggcatggagctacagtgatgcttgcatgtccctagataagacaaccagttccggccacccccatcataagaagaaaaatgatgactggaatggaaattcatttgtgagggaattgggcgaccaggcggcgcatgctaacagcatgtacgaacttggtaagtccatgaaacctgtgtatacagcagctctaaaggatgagctagtcaagccagataaggtgtacacaaaaatcaaaaagagactgctttggggcgcggaccttggcaccgtcattcgcgctgccagagcatttgggccgttctgtgaggccataaaaccccatgtaatcaaattgcctatcaaggtgggtatgaatgccatagaggatggtcctttgatttatgcagagcactccaaatataagtttcattatgatgctgactatacagcttgggactcaacacaaaacagggagatcatgatggaatccttcaacattatgtgtaagctcacagccaacccctccttggccgcagtggtggcacaggatctactctccccatctgaaatggacgttggcgactatgtgatcagtgtcaaagatggtctgccatctggctttccatgcacttcacaggtgaatag

SEQ ID NO: 2 - DNA, 658 bp, Human calicivirus; identical to SEQ ID NO: 1

SEQ ID NO: 3 - DNA, 865 bp, Human calicivirus
gtattgaacccgcatacctaggtggtaaggaccctcgagtccaaaatggtccctccctccaacaggtgctccgcgatcagctgaaaccattcgcagatcctcgtgggcgcatgcctgagcctggcttactggaggctgctgtagaaactgtgacatctatgttagaacagacaatggacaccccaagtccgtggtcttatgctgatgcctgtcaatctcttgacaagacgactagttcaggctatccccaccataaaagaaagaatgatgattggaatggcactacctttgtcagagaactcggtgaccaagcagcgcatgccaatagcatgtatgagaatgccaaacacatgaagcccatatacactgcagccctgaaggatgaattggttaagcctgagaaggtctaccagaaagttaagaaacgcctgttgtggggcgccgatcttgggacagtgatcagagctgccagggcttttggcccattctgtgatgctataaagccacatgtcatcaaattgccaataaaagttggcatgaacacaatagaggatggccctttaatttatgctgaacatgccaagtacaaaaatcattttgatgcagattacacagcatgggactctacacaaaatagacaaattatgacagaatccttttccatcatgtcacgcctcacggcctctccagaactagctgaggttgtagcccaggacttactagcaccatccgagatggatgtgggtgactatgttataagggtcaaagaaggcctaccatcaggatttccctgcacttctcaagtgaatagcataaatcactggataatcaccctttgtgcattgtctgaggctactggctcatc

SEQ ID NO: 4 - DNA, 1113 bp, Human calicivirus
aatatccccgaaccactgccccctggggtctatgaacccgcctacctcgggggcccgggaccctagggtgactggcggtccctcactccaacaagtgttgcgggatcagttaaagccatttgctgagccacgaggacgcatgccagagccaggtctcttggaggccgcagttgagactgtgacttcatcattagagcaggttatggacactcccgttccttggagctatagtgatgcgtgccagtcccttgataagaccactagttctggtttcccctaccacagaaggaagaatgacgactggaatggcaccacctttgttagggagttaggggagcaggcggcacacgctaacaacatgtatgagcaggctaagagtatgaaacccatgtacacggcagcacttaaagatgaactagtcaaaccagagaaggtataccagaaagtgaaaaagcgcttgttatggggggcagacttgggcacggtagttcgggccgcacgggcttttggcccattctgtgatgctataaaatcccacacaatcaaattgcctatcaaagttggaatgaattcaattgaggatgggccactgatctatgcagaacattcaaaatataagtaccactttgatgcagattacacagcttgggattcaactcaaaataggcaaatcatgacagagtcattttcaatcatgtgtcggctaactgcatcacctgaactagcttcagtggtggctcaagacttgctcgcaccctcagagatggatgttggcgactacgtcataagagtgaaggaaggcctcccatctggtttcccatgcacatcacaagttaatagtataaaccattggttaataactctgtgtgccctttctgaagtaactggtctgtcgccagatgttatccagtccatgtcatatttctctttctatggtgatgatgaaatagtgtcaactgacatagaatttgatccagcaaaactgacacaagttctcagagagtatggacttaaacccacccgccccgacaaaagcgagggcccaataattgtaagggaagagtgtggatgggttggtctttttacgtcgcactatctcccgcgacgccgcgg

SEQ ID NO: 5 - DNA, 620 bp, Human calicivirus
agttgtcgcgggtgtccacaccgcagcagcccgggggggcaacactgtcatatgtgccactcaagggcaagatggagaggcagtccttgagggtaatgaggacctcggcacatactgcggtgctccaatcctaggccctggcaaggcgcctaaactcagcacgaagaccaaattctggcgttcatcaccagatgccttgccacctggcacatatgaacctgcttacctgggaggcaaggaccctagagtggagaaaggaccctccctgcaacaagttatgagagatcagctaaagcccttcacagagcctaggggcaaaccacctagacctgcagtcttagaagaagctaaaaagacagtgatgaatgtcctagaacaaaccattgaccctgctaagccatggtcctactcgcaagcatgtgcctcactggacaaaaccacttctagtggtagcccccaccatgtcaagaaaaatgaccattggaatggggagtccttcactggcccccttgcagatcaagcatctaaagccaacctcatgtatgaacaggccaagcatgtgcagcccgtgtacacggccgcgctcaaagatgagcttgttaagactgacaaaatttttt

SEQ ID NO: 6 - DNA, 620 bp, Human calicivirus
tgtggttattggagtccacacggccgccgctcgtgggggaaacactgtcatatgtgccacccaggggagtgagggggaggctacacttgaaggtggtgacagtaagggaacatactgtggtgcaccaatcctaggcccaggtagtgccccaaaactcagcaccaagaccaaattctggagatcgtccacaacaccactcccacctggcacctatgaaccagcctatctcggtggtaaggatcccagagtcaagggtggcccttcattgcaacaagtcatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtctcttgacaagaccacttccagtggccatccgcaccacatgcggaaaaacgactgctggaacggggagtccttcacaggcaagctggcagaccaggcttccaaggccaacctgatgttcgaagaggggaagaacatgaccccagtctacacaggtgcgcttaaggatgagttagttaagactgacaaaatttttt

SEQ ID NO: 7 - DNA, 616 bp, Human calicivirus
tgtggttattggagtcccacggctgccgctcgtggggggaacactgtcatatgtgccacccaggggagtgagggggaggctacacttgaaggtggtgacagtaagggaacatactgtggtgcaccaatcctaggcccagggagtgccccaaaactcagcaccaagaccaaattctggagatcgtccacaacaccactcccacctggcacctatgaaccagcctatcttggtggtaaggaccccagagtcaagggtggcccttcattgcaacaagttatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtcccttgacaagaccacttccagtggccatccacaccacatgcggaaaaacgactgctggaacggggagtccttcacaggcaagctggcagaccaggcttccaaggccaacctgatgttcgaagaggggaagaacatgaccccagtctacacaggtgcgcttaagggatgagttagttaagactgacaaaatt

SEQ ID NO: 8 - DNA, 628 bp, Human calicivirus
ggggatgactacgtggtcattggggtccacacggctgctgcccgtgggggaaacactgtcatatgtgccacccagggaagtgaaggtgaggccactcttgagggcggcgatgacaaaggcacttactgtggtgccccaatcctaggtccagggagtgctccaaagcttagcaccaaaactaagttttggaggtcatccacaacaccactcccacccggtacctatgaaccagcctacctcggtggcaaggaccccaggattaaaggtggtccctcactacaacaagtcatgagggaccagctgaagccatttacagaacctagaggtaggcaaccaaaaccaagtgtattggaggctgccaagaagaccatcattaatgtacttgaacaaacaatagacccacctcaaaagtggacattctcacaggcttgtgcctcccttgacaagaccacctctagtggccacccacaccacatgagaaagaatgactgttggaatggggaatctttcacaggcaaattggcagatcaggcctcaaaggccaatttgatgtatgaggaggggaaaaatatgacaccagtttacacaggtgccctcaaggatgagctggtcaagacttgacaaaatt

SEQ ID NO: 9 - DNA, 452 bp, Human calicivirus
aaattttggagatcatccacagcaccactcccacctggtacctatgaaccagcctaccttggcggcaaggaccccagagtcaagggtggtccttcattgcaacaagttatgagggaccagctgaaaccattcactgagcccaggggcaaaccaccaaaaccaagtgtgttagaggctgccaagaaaaccatcatcaatgttcttgagcaaacaattgatccacctcaaaaatggtcattcgcgcaagcatgcgcatccctcgacaagaccacctctagtggtcacccgcaccacatgcggaaaaacgactgctggaacggggagtccttcacaggcaaattggcagaccaggcttccaaggctaacctgatgtacgaagagggaaagaacatgaccccagtttacacgggtgcgcttaaggacgaagctggtcaagactgacaaaatttttt

SEQ ID NO: 10 - DNA, 621 bp, Human calicivirus
tgtggtcattggcgtgcacaccgcagcagcacgtggaggaaacacagttatatgtgccacgcaaggaagcgagggtgaagccactcttgagggaggtgacgataagggcacatattgtggagcccccattttgggacctggtaatgcgccaaagttgagcacaaaaacaaaattctggagatcttccaacgcaccgctcccgccaggcacctatgaaccagcatacctaggtgggaaagatcaacgtgtgaagggcggtccgtctctgcaacaggtcatgagagaccaacttaaacctttcacagagcccagaggaaaaccaccaaacccgagtgttctagaatcagcaaagaagactattatcaatgttctagagcaggttattgaccccccccagaagtggtcctatgctcaggcttgtgcttcccttgacaaaacaacctccagtggacacccacaccacgttcggaagaatgattactggagtggtgaatcctttacaggaaaacttgcagaccaggcttcaaaagcaaatctcatgtatgaagaaggcaaacacatgccaccggtttacaccgccgcgctcaaggatgagctggtgaagactgacaaaattttttt

SEQ ID NO: 11 - DNA, 608 bp, Human calicivirus
gagtccacacggctgccgctcgtggggggaacactgtcatatgtgccacccaggggagtgagggggaggctacacttgaaggtggtgacagtaagggaacatactgtggtgcaccaatcctaggcccagggagtgccccaaaactcagcaccaagaccaaattctggagatcgtccacaacaccactcccacctggcacctatgaaccagcctatcttggtggtaaggaccccagagtcaagggtggcccttcattgcaacaagttatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtcccttgacaagaccacttccagtggccatccacaccacatgcggaaaaacgactgctggaacggggagtccttcacaggcaagctggcagaccaggcttccaaggccaacctgatgttcgaagaggggaagaacatgaccccagtctacacaggtgcgcttaaggatgagttagttaagactgacaaaattatt

SEQ ID NO: 12 - DNA, 603 bp, Human calicivirus
  misc_feature (465)..(465): n is selected from a, g, c, or t
gtccacacggctgccgctcgtgggggaaacactgtcatatgtgccacccaggggagtgagggggaggctacacttgaaggtggtgacagtaagggaacatactgtggtgcaccaatcctaggcccagggagtgccccaaaactcagcaccaagaccaaattctggagatcgtccacaacaccactcccacctggcacctatgaaccagcctatcttggtggtaaggaccccagagtcaagggtggcccttcattgcaacaagttatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtcccttgacaagaccacttccagtggccatccacaccacatgcggaaaaacgantgctggaacggggagtccttcacaggcaagctggcagaccaggcttccaaggccaacctgatgttcgaagaggggaagaacatgaccccagtctacacaggtgcgcttaaggatgagttagttaagactgacaaaatt

SEQ ID NO: 13 - DNA, 606 bp, Human calicivirus
gtccacacggctgccgctcgtggggggaacactgtcatatgtgccacccaggggagtgagggggaggctacacttgaaggtggtgacagtaagggaacatactgtggtgcaccaatcctaggcccagggagtgccccaaaactcagcaccaagaccaaattctggagatcgtccacaacaccactcccacctggcacctatgaaccagcctatcttggtggtaaggaccccagagtcaagggtggcccttcattgcaacaagttatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtcccttgacaagaccacttccagtggccatccacaccacatgcggaaaaacgactgctggaacggggagtccttcacaggcaagctggcagaccaggcttcaaggccaacctgatgttcgaagaggggaagaacatgaccccagtctacacaggtgcgcttaaggatgagttagttaagactgacaaaattattg

SEQ ID NO: 14 - DNA, 583 bp, Human calicivirus
acactgtcatatgtgccacccaggggagtgagggggaggctacacttgaaggtggtgacagtaagggaacatactgtggtgcaccaatcctaggcccagggagtgccccaaaactcagcaccaagaccaaattctggagatcgtccacaacaccactcccacctggcacctatgaaccagcctatcttggtggtaaggaccccagagtcaagggtggcccttcattgcaacaagttatgagggatcagttgaaaccatttacagagcccaggggcaaaccaccaaagccaagtgtgttggaagctgccaagaaaaccatcatcaatgtccttgaacaaacaatcgatccacccgagaaatggtcattcacgcaagcttgcgcgtcccttgacaagaccacttccagtggccatccacaccacatgcggaaaaacgactgctggaacggggagtccttcacaggcaagctggcagaccaggcttcaaggccaacctgatgttcgaagaggggaagaacatgaccccagtctacacaggtgcgcttaaggatgagttagttaagactgacaaaattattggaaaa

SEQ ID NO: 15 - DNA, 20 bp, ARTIFICIAL SEQUENCE
  SYNTHETIC DNA
gaggcagccgttgaaactgt

SEQ ID NO: 16 - DNA, 21 bp, ARTIFICIAL SEQUENCE
  SYNTHETIC DNA
tccttagacgccatcatcatt

SEQ ID NO: 17 - DNA, 25 bp, ARTIFICIAL SEQUENCE
  SYNTHETIC DNA
ctctcaactaaaacaaaattctgga

SEQ ID NO: 18 - DNA, 25 bp, ARTIFICIAL SEQUENCE
  SYNTHETIC DNA
catcaccatagaatgagaaatatga

SEQ ID NO: 19 - DNA, 21 bp, ARTIFICIAL SEQUENCE
  SYNTHETIC DNA
ctgcccctacatctacaagag

SEQ ID NO: 20 - DNA, 22 bp, ARTIFICIAL SEQUENCE
  SYNTHETIC DNA
tttgccataattttgtcagtct